(19) United States
(12) Patent Application Publication
(10) Pub. No.: US 2026/0207729 A1
(43) Pub. Date: Jul. 23, 2026

(54) STABILIZED TRIMERIC RSV FUSION PROTEINS WITHOUT A HETEROLOGOUS TRIMERZATION DOMAIN

(71) Applicant: MSD INTERNATIONAL BUSINESS GMBH, Lucerne (CH)

(72) Inventors: Johannes Petrus Maria Langedijk, Leiden (NL); Mark Johannes Gerardus Bakkers, Leiden (NL); Annemart Koornneef, Leiden (NL); Jaroslaw Juraszek, Leiden (NL)

(73) Assignee: MSD INTERNATIONAL BUSINESS GMBH, Lucerne (CH)

(21) Appl. No.: 19/157,873

(22) PCT Filed: Feb. 20, 2024
(86) PCT No.: PCT/EP2024/054257

§ 371 Date: Aug. 19, 2025

(30) Foreign Application Priority Data
Feb. 21, 2023 (EP) 23157769.3

(51) Int. Cl. A61K 39/12; A61P 37/04; C07K 14/005

(52) U.S. Cl. CPC A61K 39/12; A61P 37/04; C07K 14/005

(57) ABSTRACT

The present invention relates to respiratory syncytial virus (RSV) F proteins, comprising a head region and a stem region, wherein the protein comprises at least one amino acid mutation in the stem region relative to the amino acid sequence of a wild-type RSV F protein, wherein the at least one mutation in the stem region comprises a mutation of the amino acid residue at position 509 into F, I or L, and wherein amino acid positions are numbered according to the numbering of the amino acid residues in SEQ ID NO:1, and to nucleic acids encoding such proteins, as well as to uses thereof.

The present invention relates to the field of medicine. The invention in particular relates to recombinant pre-fusion RSV F proteins, to nucleic acid molecules encoding the RSV F proteins, and uses thereof, e.g. in vaccines.

### BACKGROUND OF THE INVENTION

Respiratory syncytial virus (RSV) is associated with upper and lower respiratory tract infections in humans. Worldwide, it is estimated that 64 million RSV infections occur each year resulting in 160.000 deaths (WHO Acute Respiratory Infections Update September 2009). The most severe disease occurs particularly in premature infants, the elderly and immunocompromised individuals. In children younger than 2 years, RSV is the most common respiratory tract pathogen, accounting for approximately 50% of the hospitalizations due to respiratory infections, with the peak of hospitalization occurring at 2-4 months of age. It has been reported that almost all children have been infected by RSV by the age of two. Repeated infection during lifetime is attributed to ineffective natural immunity. In the elderly, the RSV disease burden is similar to that caused by non-pandemic influenza A infections. Older adults are at particularly high risk for severe RSV-mediated disease; in the United States alone, there are an estimated 177,000 hospitalizations and 14,000 deaths due to RSV in adults aged ≥65 years. (Centers for Disease Control and Prevention. Respiratory syncytial virus infection (RSV). Trends and surveillance. https://www.cdc.gov/rsv/research/us-surveillance.html (2020). Accessed Mar. 28, 2022).

RSV is a paramyxovirus, belonging to the subfamily of Pneumoviridae. Its genome encodes for various proteins, including RSV glycoprotein (G) and RSV fusion protein (F) membrane proteins, the latter of which is the major antigenic target for neutralizing antibodies. Antibodies against the F protein can prevent virus entry into the cell and thus have a neutralizing effect.

RSV F fuses the viral and host-cell membranes by irreversible protein refolding from the labile pre-fusion conformation to the stable post-fusion conformation. Structures of both conformations have been determined for RSV F (Mclellan J S, et al. (2010, 2013, 2013); Swanson K A, et al. (2011)), as well as for the fusion proteins from related paramyxoviruses, providing insight into the complex mechanism this fusion protein undergoes. Like other class I fusion proteins, the inactive precursor, RSV F₀, requires cleavage during intracellular maturation by a furin-like protease. Cleavage of RSV F at two furin cleavage sites results in three proteins: F2, p27 and F1. The p27 fragment is not part of the mature F protein and F2 and F1 are associated by two disulfide bridges, with the F1 domain containing a hydrophobic fusion peptide (FP) at its N-terminus. In order to refold from the pre-fusion to the post-fusion conformation, the refolding region 1 (RR1) between residue 137 and 216, that includes the FP and heptad repeat A (HRA, also referred to as ‘HR1’) has to transform from an assembly of helices, loops, and strands to a long continuous helix. The FP, located at the N-terminal segment of RR1, is then able to extend away from the viral membrane and to insert into the proximal membrane of the target cell. Next, the refolding region 2 (RR2), which forms the C-terminal stem in the pre-fusion F protein and includes the heptad repeat B (HRB, also referred to as ‘HR2’), relocates to the other side of the RSV F head and binds the HRA coiled-coil trimer with the HRB domain to form the six-helix bundle. The formation of the RR1 coiled-coil and relocation of RR2 to complete the six-helix bundle are the most dramatic structural changes that occur during the refolding process.

A soluble, subunit-based vaccine requires truncation of the viral fusion protein at the C-terminus by deletion of the transmembrane (TM) and the cytoplasmic region. The remaining ectodomain of the fusion protein is considerably more labile due to removal of the membrane anchor and will either not form as a trimeric protein or even more readily refold into the post-fusion end-state. Therefore, soluble viral fusion proteins are commonly equipped with a C-terminal heterologous trimerization domain (e.g. foldon or GCN4) to restore trimer formation (Welch et al. (2012), Proc Natl Acad Sci USA, 109:16672-16677; Mclellan et al. (2013), Science, 342:592-598; Walls et al. (2016), Nat Struct Mol Biol, 23:899-905). However, addition of a non-native trimerization domain may induce non-relevant antibodies to this domain that will not cross-react with the virus and may hamper immunogenicity when the vaccine is used repeatedly and when the domain is used in other vaccines, which will increase its immunodominance.

A vaccine preventing against RSV infection is currently not yet available, but it is desired due to the high disease burden. The RSV fusion glycoprotein F is an attractive vaccine antigen as it is the principal target of neutralizing antibodies in human sera. Most neutralizing antibodies in human sera are directed against the pre-fusion conformation, but due to its instability the pre-fusion conformation has a propensity to prematurely refold into the post-fusion conformation. As indicated above, crystal structures have revealed a large conformational change between the pre-fusion and post-fusion states. The magnitude of the rearrangement suggests that only a portion of antibodies directed to the post-fusion conformation of RSV F will be able to cross react with the native conformation of the pre-fusion spike on the surface of the virus. Accordingly, efforts to produce a vaccine against RSV have focused on developing vaccines that contain or express pre-fusion forms of RSV F protein.

A need still remains for efficient vaccines against RSV, in particular vaccines based on or comprising RSV F proteins in the pre-fusion conformation. The present invention aims at providing such stable pre-fusion RSV F proteins for use in vaccinating against RSV.

### SUMMARY OF THE INVENTION

Provided herein are vaccine components that can be used to induce an immune response against RSV.

In a first aspect, the present invention provides respiratory syncytial virus (RSV) F proteins, comprising a head region and a stem region, wherein the protein comprises at least one amino acid substitution in the stem region relative to the amino acid sequence of a wild-type RSV F protein, wherein the at least one substitution in the stem region comprises a substitution of the amino acid residue at position 509 into F, I or L, and wherein amino acid positions are numbered according to the numbering of the amino acid residues in SEQ ID NO:1.

The invention further provides nucleic acids encoding the proteins according to the invention, as well as vectors comprising such nucleic acids.

Compositions comprising the protein(s), nucleic acid(s) and/or vector(s) according to the invention, as well as the use thereof are also provided.

The invention also provides isolated host cells comprising a nucleic acid according to the invention.

### BRIEF DESCRIPTION OF THE FIGURES

The foregoing summary, as well as the following detailed description of the invention, will be better understood when read in conjunction with the appended figures. It should be understood that the invention is not limited to the precise embodiments shown in the examples.

FIG. 1: RSV F forms monomers in the absence of a heterologous trimerization domain.

(A) RSV-A and (B) RSV-B F trimer (T) and monomer (M) detection in cell culture supernatant using analytical size exclusion chromatography (SEC).

FIG. 2: S509 substitutions in HR2 yield trimeric RSV-A F in the absence of a heterologous trimerization domain.

RSV-A F trimer (T) and monomer (M) detection in cell culture supernatant using analytical SEC with variations at position 509.

FIG. 3. F505W+S509F substitutions in HR2 improve trimer yield in RSV-A and RSV-B F in the absence of a heterologous trimerization domain.

(A) RSV-A F trimer detection in cell culture supernatant using analytical SEC.

(B) RSV-B F trimer (T) and monomer (M) detection in cell culture supernatant using analytical SEC.

FIG. 4. F505W+S509F substitutions in HR2 confer temperature stability to trimeric RSV-A F in the absence of a heterologous trimerization domain.

RSV-A F trimer detection in cell culture supernatant incubated 15 minutes at 4, 60, 65, and 70° C. (see legend) using analytical SEC.

FIG. 5. Head domain substitutions improve trimer yield in RSV-A and RSV-B F in the absence of a heterologous trimerization domain.

(A) RSV-A F trimer detection in cell culture supernatant using analytical SEC.

(B) RSV-B F trimer (T) and monomer (M) detection in cell culture supernatant using analytical SEC.

FIG. 6. RSV-B F trimer stability is increased by combinatorial HR2 and head domain stabilization in the absence of a heterologous trimerization domain.

(A) RSV-B F trimer (T) and monomer (M) detection in cell culture supernatant using analytical SEC at day of harvest.

(B) RSV-B F trimer (T) and monomer (M) detection in cell culture supernatant using analytical SEC after 16 days storage at 4° C.

FIG. 7. HR2 elongation improves trimer yield and stability in RSV-A and RSV-B F in the absence of a heterologous trimerization domain.

(A) RSV-A F trimer detection of short (left) and long (right) HR2 variants in cell culture supernatant using analytical SEC.

(B) RSV-A F trimer detection at day of harvest (day 0) and after two days storage at 4° C. (day 2) in cell culture supernatant using analytical SEC.

(C) RSV-B F trimer (T) and monomer (M) detection in cell culture supernatant using analytical SEC.

FIG. 8. G519V+T523I substitutions in elongated HR2 improve trimer expression in RSV-A F in the absence of a heterologous trimerization domain.

RSV-A F trimer detection in cell culture supernatant using analytical SEC.

FIG. 9. Tag-free purification and characterization of stabilized RSV-A proteins without a heterologous trimerization domain.

(A) Analytical SEC-MALS of purified RSV-A F trimers.

(B) Melting temperature (Tm50) of purified RSV-A F trimers as determined by Differential Scanning Fluorimetry (DSF). N=3 replicate measurements, and individual and average values are reported as grey and black solid lines, respectively.

(C) Trimer yield of purified RSV-A F trimers.

(D) Pre-fusion, post-fusion, and pan-specific RSV-A F antibody binding of purified RSV-A F trimers using quantitative Octet. Initial binding rate is plotted.

FIG. 10. Purification and characterization of stabilized RSV-A proteins in the presence and absence of a heterologous trimerization domain.

(A) Overview of amino acid substitutions and presence (+) or absence (−) of a foldon trimerization domain in purified RSV-A F variants.

(B) Analytical SEC-MALS of purified RSV-A F trimers.

(C) Melting temperature (Tm50) of purified RSV-A F trimers as determined by Differential Scanning Fluorimetry (DSF). N=3 replicate measurements, and individual and average values are reported as grey and black solid lines, respectively.

(D) Pre-fusion, post-fusion, and pan-specific RSV-A F antibody binding of purified RSV-A F trimers using quantitative Octet. Initial binding rate is plotted.

(E) Analytical SEC-MALS of purified RSV-A F trimers directly after purification (t=0) and after 19 weeks storage at 4° C. (t=19 weeks).

FIG. 11. Immunogenicity of stabilized RSV-A proteins in the presence and absence of a heterologous trimerization domain.

(A) Virus neutralizing antibody titers (VNT) as measured by firefly luciferase (FFL) reporter-based assay for RSV-A CL57.

(B) Foldon antibody binding titers as measured by ELISA.

(C) Prefusion RSV-F antibody binding titers as measured by ELISA.

(D) Postfusion RSV-F antibody binding titers as measured by ELISA.

(E) Ratio of prefusion/postfusion RSV-F antibody binding titers from (C) and (D).

FIG. 12. Purification, characterization, and immunogenicity of stabilized foldon-free and tagless RSV-A and RSV-B preF proteins.

(A) Analytical SEC-MALS of purified RSV F trimers.

(B) Melting temperature (Tm50) of purified RSV F trimers as determined by Differential Scanning Fluorimetry (DSF). N=3 replicate measurements, and individual and average values are reported as grey and black solid lines, respectively.

(C) Pre-fusion (CR9501, RSV90, RSD5-GL), post-fusion (ADI-15644), and pan-specific (CR9506) RSV F antibody binding of purified RSV F trimers using quantitative Octet. Initial binding rate is plotted.

(D) Analytical SEC-MALS of purified RSV F trimers directly after purification (t=0) and after 1, 3 and 6 months of storage at either 4 or 37° C.

### DETAILED DESCRIPTION OF THE INVENTION

As described above, respiratory syncytial virus, or RSV, is a respiratory virus that infects the lungs and breathing passages. RSV is the leading cause of serious viral lower respiratory tract illness in infants worldwide and an important cause of respiratory illness in the elderly.

Human RSV (HRSV) is divided into two main subtypes; HRSV-A and HRSV-B that are generally distinguished based on sequence differences in the G protein. The F proteins of A and B strains show a high degree of sequence identity (~95% in the mature ectodomain).

The F glycoprotein of human RSV is initially translated from the mRNA as a single 574-amino acid polypeptide precursor (referred to “F0” or “F0 precursor”), which contains a signal peptide sequence (amino acids 1-25) at the N-terminus. Upon translation the signal peptide is removed by a signal peptidase in the endoplasmic reticulum. The remaining portion of the F0 precursor (i.e., residues 26-574) may be further cleaved at two polybasic sites (amino acids 109/110 and 136/137) by cellular proteases (in particular furin), removing a 27-amino acid intervening sequence designated p27 (amino acids 110-136) and generating two linked fragments designated F1 (C-terminal portion; amino acids 137-574) and F2 (N-terminal portion; amino acids 26-109). F1 contains a hydrophobic fusion peptide at its N-terminus and two heptad-repeat regions (HR1 and HR2). HR1 is near the fusion peptide, and HR2 is near the TM domain. The F1 and F2 fragments are linked together through two disulfide bonds. Either the uncleaved F0 protein without the signal peptide sequence or a F1-F2 heterodimer can form an RSV F protomer. Three such protomers assemble to form the final RSV F protein complex, which is a homotrimer of the three protomers.

As described above, a vaccine against RSV infection is currently not yet available. One potential approach to producing a vaccine is providing a subunit vaccine based on soluble purified RSV F protein. However, for this approach it is desirable that the purified RSV F protein is in a conformation which resembles the conformation of the pre-fusion state of RSV F protein, and which is stable over time, i.e. which remains in the pre-fusion conformation, e.g. as determined by specific binding of the RSV F protein to antibodies that are specific for the pre-fusion conformation to the RSV F protein, and can be produced in sufficient quantities. In addition, for a soluble, subunit-based protein vaccine, the RSV F protein needs to be truncated by deletion of the transmembrane (TM) and the cytoplasmic region to create a soluble secreted F protein (sF protein). Because the TM region is responsible for membrane anchoring and increases stability, the anchorless soluble F protein is considerably more labile than the full-length protein and will even more readily refold into the post-fusion end-state.

In order to obtain F protein in the stable pre-fusion conformation that shows high expression levels and high stability, the pre-fusion conformation thus needs to be stabilized. Because also the full-length (membrane-bound) RSV F protein is metastable, stabilization of the pre-fusion conformation is also desirable for the full-length RSV F protein, i.e. including the TM and cytoplasmic region, e.g. for any live attenuated, genetic immunization or vector-based vaccine approach.

The present invention provides respiratory syncytial virus (RSV) F proteins, comprising a head region and a stem region, wherein the protein comprises at least one amino acid substitution in the stem region relative to the amino acid sequence of a wild-type RSV F protein, wherein the at least one substitution in the stem region comprises a substitution of the amino acid residue at position 509 into F, I or L, and wherein amino acid positions are numbered according to the numbering of the amino acid residues in SEQ ID NO:1. According to the present invention, it has been shown that RSV F proteins comprising the at least one substitution at position 509 are less fusogenic (i.e. have a reduced fusogenicity), as compared to RSV F proteins without a substitution at position 509, which may result in a reduced reactogenicity when used as a vaccine component.

In addition, it has been shown that stable trimeric RSV F ectodomains can be obtained when the amino acid residue at position 509 has been substituted into F, I or L.

As used herein the head domain (comprising amino acids 27-490) is defined as the RSV F ectodomain without the signal peptide and without the HR2 region. The stem domain is defined here as the HR2 region (i.e. comprising amino acids 491-524).

The present invention thus provides trimeric recombinant pre-fusion RSV F proteins with a reduced fusogenicity.

In addition, or alternatively, the present invention provides trimeric RSV F proteins that are stabilized in the pre-fusion conformation. In the research that led to the present invention, several modifications, e.g. mutations (substitutions) of amino acids as compared to the amino acid sequence of a wild-type RSV F protein, in particular the amino acid sequence of SEQ ID NO: 1, were introduced in order to obtain said stable trimeric pre-fusion RSV F proteins. The stable pre-fusion RSV F proteins of the invention are in the pre-fusion conformation, i.e. they comprise (display) at least one epitope that is specific to the pre-fusion conformation F protein. An epitope that is specific to the pre-fusion conformation F protein is an epitope that is not present in the post-fusion conformation. Without wishing to be bound by any particular theory, it is believed that the pre-fusion conformation of RSV F protein may contain epitopes that are the same as those present on the RSV F protein expressed on natural RSV virions, and therefore may provide advantages for eliciting protective neutralizing antibodies. In certain embodiments, the proteins of the invention comprise at least one epitope that is recognized by a pre-fusion specific anti-RSV monoclonal antibody. An example of such pre-fusion-specific RSV F antibody is RSV90 (Mousa et al. (2017) Nat Microbiol, 2:16271).

In certain preferred embodiments, the recombinant pre-fusion RSV F proteins comprise at least one epitope that is recognized by at least one pre-fusion specific monoclonal antibody, as described above, and are trimeric.

In certain embodiments, the protein comprises at least one amino acid substitution in the head region relative to the amino acid sequence of a wild-type RSV F protein and at least one amino acid substitution in the stem region, wherein the at least one mutation in the stem region comprises a substitution of the amino acid residue at position 509 into F, I or L.

In certain embodiments, the at least one amino acid mutation in the head region is a substitution of the amino at residue 215 into P or a substitution of the amino acid residue at position 486 into N. According to the invention it has been shown that these mutations stabilize the pre-fusion conformation, increase trimer yield and/or trimer stability.

In certain embodiments, the proteins comprise at least one additional substitution in the head region. Adding one or more specific mutations in the head region increases trimer yield and/or trimer stability and/or pre-fusion conformation stability.

In a further embodiment, the protein comprises at least two amino acid substitutions in the head region relative to the amino acid sequence of a wild-type RSV F protein and at least one amino acid substitution in the stem region, wherein the at least one substitution in the stem region comprises a substitution of the amino acid residue at position 509 into F, I or L.

In certain embodiments, the at least two amino acid mutations in the head region comprise a substitution of the amino at residue 215 into P and a substitution of the amino acid residue at position 486 into N.

In particular embodiments, the proteins comprise at least three amino acid substitutions in the head region relative to the amino acid sequence of a wild-type RSV F protein and at least one amino acid substitution in the stem region, wherein the at least one substitution in the stem region comprises a substitution of the amino acid residue at position 509 into F, I or L. According to the invention, it has been shown that proteins comprising at least three substitutions in the head region in combination with the substitution of the amino acid at position 509, have an increased trimer yield and/or trimer stability and/or pre-fusion conformation stability.

According to the invention, the RSV F protein may be an F protein of an RSV A subtype or an F protein of an RSV B subtype. Thus, according to the invention, the substitutions may be introduced into an F protein of an RSV A subtype, such as an RSV F protein comprising the amino acid sequence of SEQ ID NO: 1. In an alternative embodiment, the substitutions may be introduced into an F protein of an RSV B subtype, such as an RSV F protein comprising the amino acid sequence of SEQ ID NO: 40.

In certain embodiments, in particular when the RSV F protein is from an RSV A subtype, the at least one additional mutation in the head region is selected from the group consisting of a mutation of the amino acid residue at position 328 into P, a mutation of the amino acid residue at position 354 into L, a mutation of the amino acid at position 487 into L, a mutation of the amino acid residue at position 489 into Y, a mutation of the amino acid residue at position 494 into I, a mutation of the amino acid residue at position 519 into V and a mutation of the amino acid residue 523 into I.

In addition, or alternatively, the amino acid residue at position 101 is not P. Preferably, the amino acid residue at position 101 is Q, S, T, A or G.

In further embodiment, in particular when the RSV F protein is from an RSV B subtype, the at least one additional mutation in the head region is selected from the group consisting of a mutation of the amino acid residue at position 152 into M, a mutation of the amino acid residue at position 203 into I, a mutation of the amino acid residue at position 354 into L, a mutation of the amino acid at position 487 into L, a mutation of the amino acid residue at position 489 into Y.

In addition, or alternatively, the amino acid residue at position 101 is not P. Preferably, the amino acid residue at position 101 is Q, S, T, A or G.

In preferred embodiments, the at least one mutation of the amino acid residue in the stem region at position 509 is a mutation into F. The presence of an F at position 509 has been shown to increase trimer yield and/or trimer stability.

In certain embodiments, the proteins further comprise a second mutation in the stem region, wherein said second mutation is a mutation of the amino acid residue at position 505 into W. The presence of second mutation at position 505 has been shown to increase trimer yield and/or trimer stability.

In certain preferred embodiments, the proteins comprise a substitution of the amino acid residue at position 509 into F, I or L, preferably F, and a mutation of the amino acid residue at position 505 into W.

In certain embodiments, the proteins comprise at least two amino acid substitutions in the head region relative to the amino acid sequence of a wild-type RSV F protein, in particular the amino acid sequence of SEQ ID NO: 1 or SEQ ID NO: 40, and a substitution of the amino acid residue at position 509 into F, I or L, preferably F, and a substitution of the amino acid residue at position 505 into W.

According to the invention, it has been shown that trimer expression (trimer yield) is increased as compared to RSV F proteins without the substitution(s) of the invention.

In addition, or alternatively, the trimer stability is increased as compared to RSV F proteins without the substitution(s) of the invention.

In certain embodiments, the RSV F proteins are trimeric after 15-minutes incubation at 65° C.

In addition, or alternatively, the proteins are trimeric after storage for at least 2 days at 4° C., in particular the proteins are trimeric after storage for at least 16 days at 4° C., preferably for at least 3, 4, 5, 6, 7, 8, 9, 10, 11, 12, 13, 14, 15, 16, 17, 18, or 19 weeks at 4° C.

In addition, or alternatively, the RSV F proteins have a higher melting temperature as compared to RSV F proteins without the substitution(s) of the invention.

As used herein, a mutation refers to a change (modification or substitution) from one amino acid into a different amino acid in a protein. The terms amino acid mutation and substitution are used interchangeably throughout this application. As used throughout the present application, the position of the amino acid residues (or amino acid positions) are given in reference to the sequence of the RSV F protein of SEQ ID NO: 1. As used in the present invention, the wording “the amino acid residue at position e.g. 509 of the RSV F protein thus means the amino acid corresponding to the amino acid at position 509 in the RSV F protein of SEQ ID NO: 1. It is noted that, in the numbering system used throughout this application 1 refers to the N-terminal amino acid of an immature F0 protein (SEQ ID NO:1), i.e. including the signal peptide. When an RSV strain other than the RSV-A strain of SEQ ID NO: 1 is used, e.g. an RSV B strain, the amino acid positions of the F protein are to be numbered with reference to the numbering of the F protein of SEQ ID NO: 1, by aligning the sequences with the F protein of SEQ ID NO: 1 with the insertion of gaps as needed. Sequence alignments can be done using methods well known in the art, e.g. by CLUSTALW, Bioedit or CLC Workbench.

An amino acid according to the invention can be any of the twenty naturally occurring (or ‘standard’ amino acids) or variants thereof, such as e.g. D-amino acids (the D-enantiomers of amino acids with a chiral center), or any variants that are not naturally found in proteins, such as e.g. norleucine. The standard amino acids can be divided into several groups based on their properties. Important factors are charge, hydrophilicity or hydrophobicity, size and functional groups. These properties are important for protein structure and protein-protein interactions. Some amino acids have special properties such as cysteine, that can form covalent disulfide bonds (or disulfide bridges) to other cysteine residues, proline that induces or stabilizes turns of the protein backbone, and glycine that is more flexible than other amino acids. Table 1 shows the abbreviations and properties of the standard amino acids. It will be appreciated by a skilled person that the mutations can be made to the protein by routine molecular biology procedures.

In certain embodiments, the stable pre-fusion RSV F proteins according to the invention are soluble proteins, i.e. RSV F protein ectodomains. Thus, in certain embodiments, the RSV F proteins (in particular the F1 domain) have been truncated (i.e. the transmembrane and cytoplasmic region have been (partially) deleted). In certain embodiments, the RSV F protein has been truncated after amino acid residue 513.

In certain preferred embodiments, the RSV F protein has been truncated after the amino acid residue 524. According to the invention, it has been shown that a longer HR2 may increase the stability of the RSV F proteins. Thus, according to the invention it has been shown that a longer HR2 results in an increase in the melting temperature.

In certain preferred embodiments, the RSV F proteins have been truncated after the amino acid at position 524, comprise at least three amino acid mutations in the head region relative to the amino acid sequence of a wild-type RSV F protein and at least one amino acid mutation in the stem region, wherein the at least one mutation in the stem region comprises a mutation of the amino acid residue at position 509 into F, I or L, preferably wherein the amino acid residue at position 509 is F.

In certain other preferred embodiments, the RSV F proteins are RSV F proteins from an RSV A subtype, have been truncated after the amino acid at position 524, comprise at least three amino acid mutations in the head region relative to the amino acid sequence of a wild-type RSV F protein and at least one amino acid mutation in the stem region, wherein the at least one mutation in the stem region comprises a mutation of the amino acid residue at position 509 into F, I or L, preferably wherein the amino acid residue at position 509 is F.

In certain embodiments, the proteins are from an RSV B subtype, have been truncated after the amino acid at position 524, comprise at least three amino acid substitutions in the head region relative to the amino acid sequence of a wild-type RSV F protein and a substitution of the amino acid residue at position 509 into F, I or L, preferably F, and substitution of the amino acid residue at position 505 into W.

As used herein a “truncated” protein refers to an RSV F protein that is not a full-length protein, i.e. a protein wherein C-terminally one or more amino acid residues have been deleted. In certain embodiments, at least the transmembrane domain and cytoplasmic domain have been deleted to permit expression as a soluble ectodomain. Because the TM region is responsible for membrane anchoring and trimerization, the “anchorless” (i . . . e without TM and cytoplasmic domain) soluble F protein is monomeric and shows low expression. In order to obtain soluble trimeric F protein in the stable pre-fusion conformation, the pre-fusion conformation thus needs to be stabilized. In order to promote trimerization it is known to replace the TM/CT region with a heterologous trimerization domain, such as a foldon domain.

In certain preferred embodiments, the truncated RSV F proteins do not comprise a heterologous trimerization domain. According to the present invention, soluble trimeric RSV pre-fusion F proteins can be provided, without a heterologous trimerization domain. According to the invention, it has surprisingly been found that by the presence of the one or more of the stabilizing mutations in the HR2 (or stem domain), the trimer content is increased, even if no heterologous trimerization domain is present, as compared to RSV F proteins without the one or more stabilizing mutations in the HR2 domain.

In certain embodiments, the proteins comprise an amino acid sequence selected from the group consisting of SEQ ID NO: 9, 11, 13, 17-29, 34, 38-39, and 43-44 or an amino acid sequence with at least 90%, at least 95%, at least 97%, at least 98% or at least 99% amino acid sequence identity, or a fragment thereof. In certain preferred embodiments, the protein comprise an amino acid sequence consisting of SEQ ID NO: 43 or a fragment thereof or having at least 90%, at least 95%, at least 97%, at least 98% or at least 99% amino acid sequence identity with SEQ ID NO: 43. In certain preferred embodiments, the protein comprise an amino acid sequence consisting of SEQ ID NO: 44 or a fragment thereof or having at least 90%, at least 95%, at least 97%, at least 98% or at least 99% amino acid sequence identity with SEQ ID NO: 44.

Fragments of the RSV F proteins as described herein are also encompassed by the present invention. The fragment may result from either or both of amino-terminal (e.g. by cleaving off the signal sequence) and carboxy-terminal deletions (e.g. by (partially) deleting the transmembrane region and/or cytoplasmic tail). The fragment may be chosen to comprise an immunologically active fragment of the F protein, i.e. a part that will give rise to an immune response in a subject. This can be easily determined using in silico, in vitro and/or in vivo methods, all routine to the skilled person.

In certain embodiments, the proteins according to the invention do not comprise a signal sequence, also referred to as leader sequence or signal peptide, corresponding to amino acids 1-26 of SEQ ID NO: 1. Signal sequences typically are short (e.g. 5-30 amino acids long) amino acid sequences present at the N-terminus of the majority of newly synthesized proteins that are destined towards the secretory pathway, and are typically cleaved by signal peptidase to generate a free signal peptide and a mature protein.

In certain embodiments, the amino acids 110-136 (i.e. the p27 peptide) are absent from the RSV F protein. Although furin typically cleaves between the amino acid position 109 and 110, it has been shown that in certain embodiments, the C-terminal arginine residue(s) at the F2 domain are cleaved out in the mature protein. Thus, in certain embodiments, the amino acids 109-136 or 108-136 are absent from the RSV F protein.

In certain embodiments, the proteins comprise a His-Tag, Strep-tag or C-tag. A His-Tag or polyhistidine-tag is an amino acid motif in proteins that consists of at least five histidine (H) residues; a Strep-tag is an amino acid sequence that consist of 8 residues (WSHPQFEK (SEQ ID NO: 41)); a C-tag is an amino acid motif that consists of 4 residues (EPEA; SEQ ID NO: 42). The tags are often at the N- or C-terminus of the protein and are generally used for purification purposes. In preferred embodiments, the proteins do not comprise such N- or C-terminal tags.

The present invention further provides nucleic acids encoding the proteins as described herein. Such nucleic acids may e.g. be used to produce the proteins according to the invention, e.g. in recombinant protein expression systems. In addition, nucleic acid vaccines represent another vaccine approach where synthetic sequences are used to express antigenic peptides or proteins in situ. Genetic immunization may promote superior adaptive immunity by activating both humoral and cell-mediated responses and has manufacturing advantages over traditional vaccines.

In preferred embodiments, the nucleic acid molecules encoding the proteins according to the invention are codon-optimized for expression in mammalian cells, preferably human cells. Methods of codon-optimization are known and have been described previously (e.g. WO 96/09378). A sequence is considered codon-optimized if at least one non-preferred codon as compared to a wild-type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

It will be understood by a skilled person that numerous different polynucleotides and nucleic acid molecules can encode the same protein as a result of the degeneracy of the genetic code. It is also understood that skilled persons may, using routine techniques, make nucleotide substitutions that do not affect the protein sequence encoded by the nucleic acid molecules to reflect the codon usage of any particular host organism in which the proteins are to be expressed. Therefore, unless otherwise specified, a “nucleotide sequence or nucleic acid molecule encoding an amino acid sequence” includes all nucleotide sequences or nucleic acid molecules that are degenerate versions of each other and that encode the same amino acid sequence. Nucleotide sequences that encode proteins and RNA may or may not include introns.

In certain embodiment, the nucleic acid molecules according to the invention encode a protein comprising an amino acid sequence selected from the group consisting of SEQ ID NO: 9, 11, 13, 17-29, 34, 38-39 and 43-44 or a fragment thereof.

Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScripts, Invitrogen, Eurofins).

The nucleic acids according to the invention may be DNA or RNA.

In certain embodiments, the RNA is mRNA, modified mRNA, self-replicating RNA, or circular mRNA. RNA vaccines as provided herein may have superior properties in that they produce much larger antibody titers and produce responses earlier than anti-viral therapeutic treatments. While not wishing to be bound by theory, it is believed that RNA vaccines are better designed to produce the appropriate protein conformation upon translation, as the RNA vaccines co-opt natural cellular machinery. Unlike traditional vaccines, which are manufactured ex vivo and may trigger unwanted cellular responses, RNA vaccines as provided herein are presented to the cellular system in a more native fashion.

The present invention thus also provides RSV vaccines that include (i) at least one ribonucleic acid (RNA) polynucleotide having an open reading frame encoding at least one RSV F protein as described herein or an immunogenic fragment thereof (e.g. an immunogenic fragment capable of raising an immune response to RSV), and (ii) a pharmaceutically acceptable carrier. In certain embodiment, the nucleic acid molecules are RNA polynucleotides having an open reading frame encoding an RSV F protein according to the invention, formulated in a cationic lipid nanoparticle.

The invention also provides vectors comprising a nucleic acid molecule as described above. In certain embodiments, a nucleic acid molecule according to the invention thus is part of a vector. In certain embodiments of the invention, the vector is an adenovirus vector.

Host cells comprising the nucleic acid molecules encoding the pre-fusion RSV F proteins form also part of the invention. The pre-fusion RSV F proteins may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, fungi, insect cells, and the like, or transgenic animals or plants. In certain embodiments, the cells are from a multicellular organism, in certain embodiments they are of vertebrate or invertebrate origin. In certain embodiments, the cells are mammalian cells. In certain embodiments, the cells are human cells. In general, the production of a recombinant proteins, such the pre-fusion RSV F proteins of the invention, in a host cell comprises the introduction of a heterologous nucleic acid molecule encoding the protein in expressible format into the host cell, culturing the cells under conditions conducive to expression of the nucleic acid molecule and allowing expression of the protein in said cell. The nucleic acid molecule encoding a protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed.

Cell culture media are available from various vendors, and a suitable medium can be routinely chosen for a host cell to express the protein of interest, here the pre-fusion RSV F proteins. The suitable medium may or may not contain serum.

A “heterologous nucleic acid molecule” (also referred to herein as ‘transgene’) is a nucleic acid molecule that is not naturally present in the host cell. It is introduced into for instance a vector by standard molecular biology techniques. A transgene is generally operably linked to expression control sequences. This can for instance be done by placing the nucleic acid encoding the transgene(s) under the control of a promoter. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt. −735 to +95 from the CMV immediate early gene enhancer/promoter. A polyadenylation signal, for example the bovine growth hormone polyA signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, polyA sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)).

The invention further provides pharmaceutical compositions comprising a pre-fusion RSV F protein, and/or fragment thereof, and/or a nucleic acid molecule, and/or a vector, as described herein. The invention thus provides compositions comprising a pre-fusion RSV F protein, or fragment thereof, that displays an epitope that is present in a pre-fusion conformation of the RSV F protein but is absent in the post-fusion conformation. The invention also provides compositions comprising a nucleic acid molecule and/or a vector, encoding such pre-fusion RSV F protein or fragment. The invention in particular provides pharmaceutical compositions, e.g. vaccine compositions, comprising a pre-fusion RSV F protein, a RSV F protein fragment, and/or a nucleic acid molecule, and/or a vector, as described above and one or more pharmaceutically acceptable excipients.

The invention also provides the use of a stabilized pre-fusion RSV F protein (fragment), a nucleic acid molecule, and/or a vector, according to the invention, for vaccinating a subject against RSV.

The invention also provides the use of a stabilized pre-fusion RSV F protein (fragment), a nucleic acid molecule, and/or a vector, according to the invention for inducing an immune response against RSV F protein in a subject. Further provided are methods for inducing an immune response against RSV F protein in a subject, comprising administering to the subject a pre-fusion RSV F protein (fragment), and/or a nucleic acid molecule, and/or a vector, according to the invention. Further provided is the use of the pre-fusion RSV F protein (fragments), and/or nucleic acid molecules, and/or vectors according to the invention for the manufacture of a medicament for use in inducing an immune response against RSV F protein in a subject. The immune response is typically characterized by an increase in antibodies against the RSV F protein.

The invention in particular provides pre-fusion RSV F protein (fragments), and/or nucleic acid molecules, and/or vectors according to the invention for use as a vaccine.

The pre-fusion RSV F protein (fragments), nucleic acid molecules, or vectors of the invention may be used for prevention (prophylaxis) and/or treatment of RSV infections. In certain embodiments, the prevention and/or treatment may be targeted at patient groups that are susceptible RSV infection. Such patient groups include, but are not limited to e.g., the elderly (e.g. ≥50 years old, ≥60 years old, and preferably ≥65 years old), the young (e.g. ≤5 years old, ≤1 year old), pregnant women (for maternal immunization), and hospitalized patients and patients who have been treated with an antiviral compound but have shown an inadequate antiviral response.

The pre-fusion RSV F proteins, fragments, nucleic acid molecules and/or vectors according to the invention may be used in stand-alone treatment and/or prophylaxis of a disease or condition caused by RSV, or in combination with other prophylactic and/or therapeutic treatments, such as (existing or future) vaccines, antiviral agents and/or monoclonal antibodies.

The invention further provides methods for preventing and/or treating RSV infection in a subject utilizing the pre-fusion RSV F proteins or fragments thereof, nucleic acid molecules and/or vectors according to the invention. In a specific embodiment, a method for preventing and/or treating RSV infection in a subject comprises administering to a subject in need thereof an effective amount of a pre-fusion RSV F protein (fragment), nucleic acid molecule and/or a vector, as described above. A therapeutically effective amount refers to an amount of a protein, nucleic acid molecule or vector, that is effective for preventing, ameliorating and/or treating a disease or condition resulting from infection by RSV. Prevention encompasses inhibiting or reducing the spread of RSV or inhibiting or reducing the onset, development or progression of one or more of the symptoms associated with infection by RSV. Amelioration as used in herein may refer to the reduction of visible or perceptible disease symptoms, viremia, or any other measurable manifestation of RSV infection.

For administering to subjects, such as humans, the invention may employ pharmaceutical compositions comprising a pre-fusion RSV F protein (fragment), a nucleic acid molecule and/or a vector as described herein, and a pharmaceutically acceptable carrier or excipient. In the present context, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The RSV F proteins, or nucleic acid molecules, preferably are formulated and administered as a sterile solution although it may also be possible to utilize lyophilized preparations. Sterile solutions are prepared by sterile filtration or by other methods known per se in the art. The solutions are then lyophilized or filled into pharmaceutical dosage containers. The pH of the solution generally is in the range of pH 3.0 to 9.5, e.g. pH 5.0 to 7.5. The RSV F proteins typically are in a solution having a suitable pharmaceutically acceptable buffer, and the composition may also contain a salt. Optionally stabilizing agent may be present, such as albumin. In certain embodiments, detergent is added. In certain embodiments, the RSV F proteins may be formulated into an injectable preparation.

In certain embodiments, a composition according to the invention further comprises one or more adjuvants. Adjuvants are known in the art to further increase the immune response to an applied antigenic determinant. The terms “adjuvant” and “immune stimulant” are used interchangeably herein and are defined as one or more substances that cause stimulation of the immune system. In this context, an adjuvant is used to enhance an immune response to the RSV F proteins of the invention. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc), which stimulate immune response upon interaction with recipient cells. In certain embodiments the compositions of the invention comprise aluminium as an adjuvant, e.g. in the form of aluminium hydroxide, aluminium phosphate, aluminium potassium phosphate, or combinations thereof, in concentrations of 0.05 −5 mg, e.g. from 0.075-1.0 mg, of aluminium content per dose.

In other embodiments, the compositions do not comprise adjuvants.

In certain embodiments, the invention provides methods for making a vaccine against respiratory syncytial virus (RSV), comprising providing an RSV F protein (fragment), nucleic acid or vector according to the invention and formulating it into a pharmaceutically acceptable composition. The term “vaccine” refers to an agent or composition containing an active component effective to induce a certain degree of immunity in a subject against a certain pathogen or disease, which will result in at least a decrease (up to complete absence) of the severity, duration or other manifestation of symptoms associated with infection by the pathogen or the disease. In the present invention, the vaccine comprises an effective amount of a pre-fusion RSV F protein (fragment) and/or a nucleic acid molecule encoding a pre-fusion RSV F protein, and/or a vector comprising said nucleic acid molecule, which results in an effective immune response against RSV. This provides a method of preventing serious lower respiratory tract disease leading to hospitalization and the decrease in frequency of complications such as pneumonia and bronchiolitis due to RSV infection and replication in a subject. The term “vaccine” according to the invention implies that it is a pharmaceutical composition, and thus typically includes a pharmaceutically acceptable diluent, carrier or excipient. It may or may not comprise further active ingredients. In certain embodiments it may be a combination vaccine that further comprises other components that induce an immune response, e.g. against other proteins of RSV and/or against other infectious agents, e.g. against HMPV, PIV and/or influenza. The administration of further active components may for instance be done by separate administration or by administering combination products of the vaccines of the invention and the further active components.

In addition, the proteins of the invention may be used as diagnostic tool, for example to test the immune status of an individual by establishing whether there are antibodies in the serum of such individual capable of binding to the protein of the invention. The invention thus also relates to an in vitro diagnostic method for detecting the presence of an RSV infection in a patient said method comprising the steps of a) contacting a biological sample obtained from said patient with a protein according to the invention; and b) detecting the presence of antibody-protein complexes.

The invention is further illustrated in the following examples. The examples do not limit the invention in any way. They merely serve to clarify the invention.

### EXAMPLES

### Example 1. Instability of Soluble RSV F Ectodomain Protein

Plasmids coding for recombinant RSV-A and RSV-B F protein ectodomains truncated after amino acid 513 according to SEQ ID NO: 1 were synthesized and codon-optimized at Genscript. The constructs were cloned into pCDNA2004 by standard methods widely known within the field involving site-directed mutagenesis and PCR and sequenced. RSV-A and RSV-B F ectodomains were designed both with and without a foldon trimerization domain. Additionally, RSV-A F carried N67I, S215P and D486N substitutions, and RSV-B F carried P101Q, I152M, L203I, S215P, D486N, and D489Y substitutions. Proteins were expressed in Expi293F cells using ExpiFectamine (Life Technologies) according to the manufacturer's instructions and cultured for 3 days at 37° C. and 10% CO₂. To assess RSV F trimer expression, sterile-filtered crude cell culture supernatant was applied to a Unix-C SEC-300 15 cm column (Sepax Technologies) with the corresponding guard column (Sepax Technologies) equilibrated in running buffer (150 mM sodium phosphate, 50 mM NaCl, pH 7.0) at 0.35 mL/min. Analytical SEC data was analyzed using Chromeleon 7.2.8.0 software package. OD280 values were displayed after subtraction of OD280 signal of mock-transfected cells.

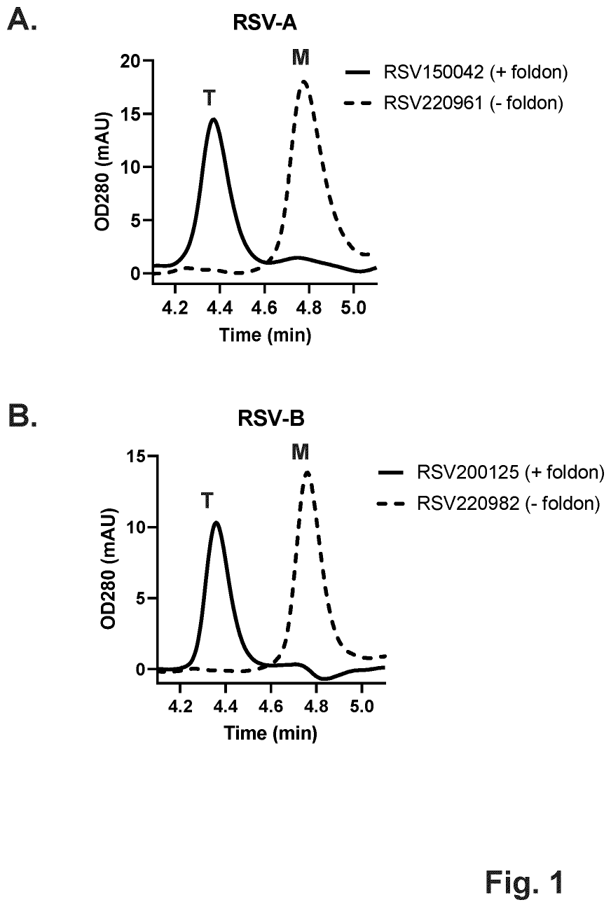

RSV-A and RSV-B F trimer with a foldon trimerization domain eluted at approximately 4.4 minutes retention time (RSV150042 and RSV200125, respectively). No trimer was detected upon expression of RSV-A and RSV-B F ectodomain without a foldon trimerization domain (RSV220961 and RSV220982, respectively) (FIG. 1). Removal of the trimerization domain instead yielded a monomer peak eluting at approximately 4.8 minutes retention time, illustrating the instability of both RSV-A and RSV-B F ectodomain trimers.

### Example 2. Stabilizing Mutations in HR2 Improve Trimerization of RSV-A and RSV-B F in the Absence of a Heterologous Trimerization Domain

To stabilize the HR2 region (amino acids 491-524) of RSV F and to allow RSV F ectodomain trimer expression in the absence of a heterologous trimerization domain, amino acid residue S509 was substituted in the stem region of RSV-A F by a hydrophobic amino acid (RSV220963-RSV220968).

Recombinant RSV-A F protein ectodomains did not have a heterologous trimerization domain and were truncated after amino acid 524 and additionally carried N67I, S215P and D486N substitutions. Trimer expression was assessed three days after transfection in Expi293F cells, as described in example 1.

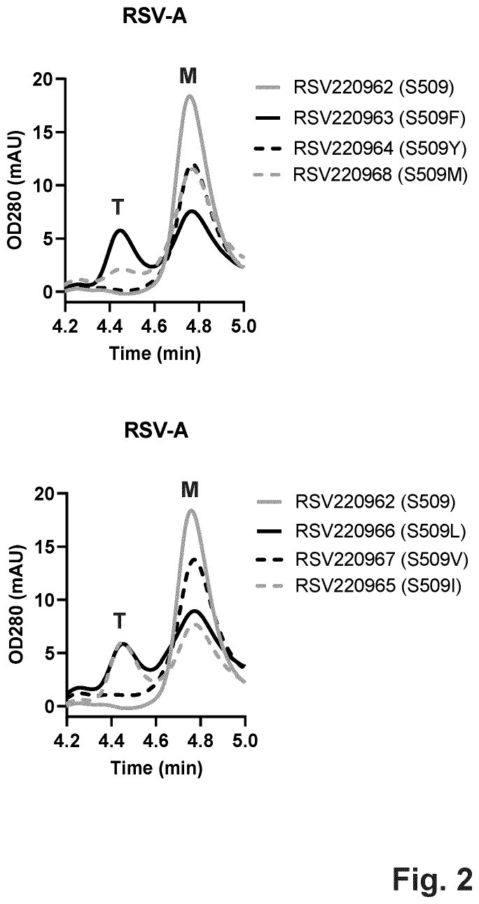

RSV220962 without a trimerization domain and with N67I, S215P, D486N, and wild-type S509 eluted as a monomeric peak at approximately 4.8 minutes retention time, as did S509Y and S509V substitutions (RSV220964, RSV220967) (FIG. 2). A minimal trimeric peak eluting at approximately 4.4 minutes retention time was detected for S509M (RSV220968), and a more pronounced trimer peak was measured by substitutions S509F, S509I and S509L (RSV220963, RSV220965, RSV220966) (FIG. 2).

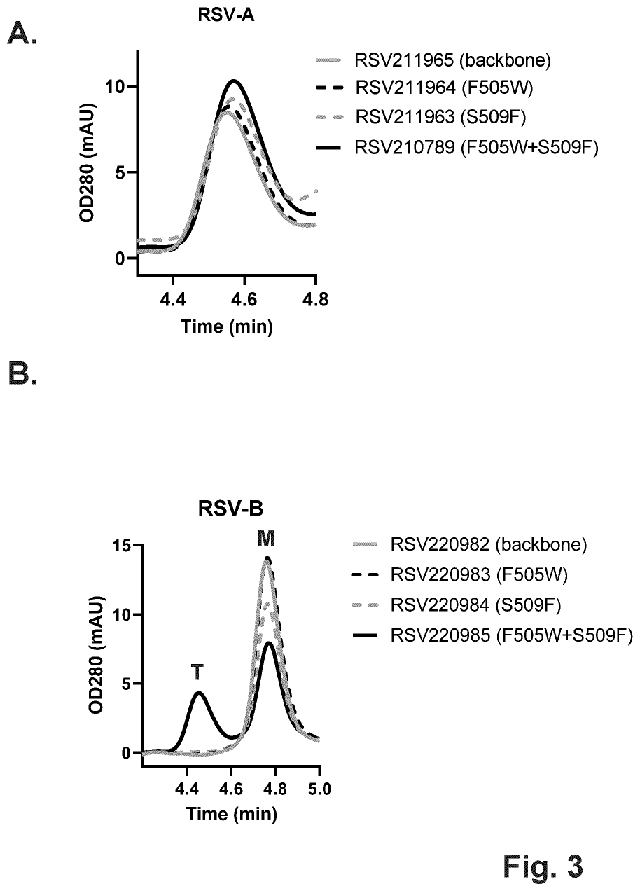

The S509F substitution was subsequently combined with another amino acid substitution in the HR2 region of RSV F at position F505, and their single and combinatorial effect was assessed in RSV-A and RSV-B F ectodomain variants without a heterologous trimerization domain (FIG. 3). To this end, plasmids coding for recombinant RSV-A F protein ectodomains truncated after amino acid 513 and additionally carrying a C-tag and P101Q, S215P, Q354L, D486N, E487L, and D489Y substitutions were employed. For RSV-B F, protein ectodomains truncated after amino acid 513 and additionally carrying P101Q, I152M, L203I, S215P, D486N, and D489Y substitutions were employed. Trimer expression was assessed three days after transfection in Expi293F cells, as described in example 1.

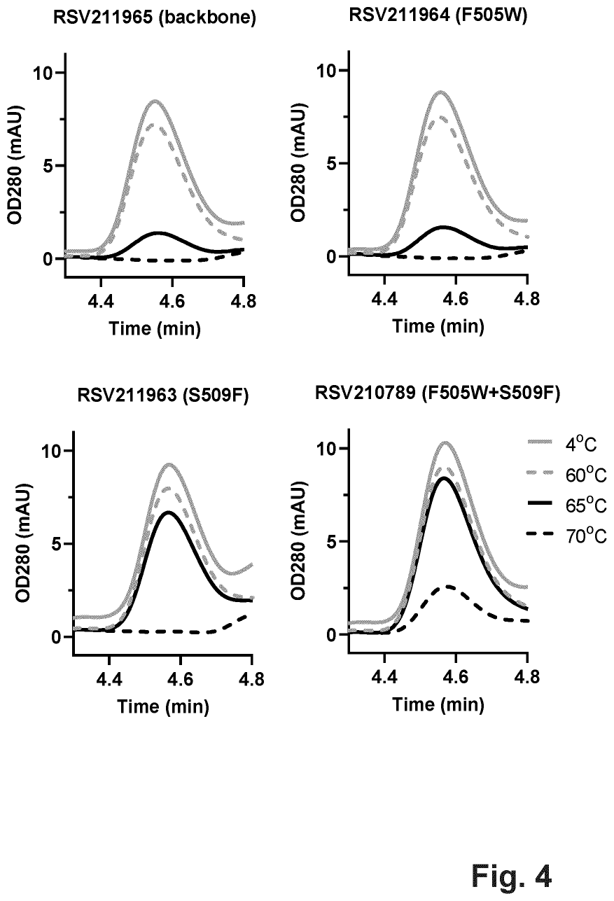

The RSV-A F backbone RSV211965 without HR2 substitutions and without a heterologous trimerization domain eluted as a trimer peak at approximately 4.6 minutes retention time (FIG. 3A). Trimeric RSV-A F ectodomain RSV211965 eluted later than RSV-A F variants described in FIG. 1 because of the shorter ectodomain employed in this variant. Introduction of single substitutions F505W (RSV211964) and S509F (RSV211963) had a minimal effect on trimer yield, while the combined introduction of F505W+S509F (RSV210789) demonstrated a more pronounced increase in RSV-A F trimer yield, accompanied by a shift to a longer retention time, indicative of a more closed conformation of the stabilized variant (FIG. 3A). Moreover, introduction of S509F, and in particular the combination of F505W+S509F increased trimer stability as demonstrated by minimal loss of RSV-A F trimer peak after 15-minutes incubation at 65° C. (FIG. 4).

The RSV-B F backbone RSV220982 without HR2 substitutions and without a heterologous trimerization domain eluted as a monomer peak at approximately 4.8 minutes retention time (FIG. 3B). Substitution with either F505W (RSV220983) or S509F (RSV220984) single mutations similarly expressed monomer and did not yield trimer expression. However, the combined introduction of F505W+S509F reduced monomer content and resulted in detectable trimer expression (RSV220985, FIG. 3B).

In summary, HR2 stabilization by F505W and S509F substitutions improved RSV F ectodomain trimer expression and stability.

### Example 3. Stabilizing Head Domain Mutations Improve Trimerization of RSV-A and RSV-B F in the Absence of a Heterologous Trimerization Domain

To stabilize the head domain (amino acids 27-490) of RSV F and to allow RSV F ectodomain trimer expression in the absence of a heterologous trimerization domain, various amino acid substitutions were evaluated for improved trimer expression. Plasmids were made coding for recombinant RSV-A and RSV-B F protein ectodomains that did not have a heterologous trimerization domain and were truncated after amino acid 513. RSV-A F variants carried a C-tag and P101Q, D486N, F505W and S509F substitutions (RSV210776). RSV-B F variants carried P101Q, 1152M, L203I, S215P, D486N, and D489Y (RSV220982). Trimer expression was assessed three days after transfection in Expi293F cells, as described in example 1.

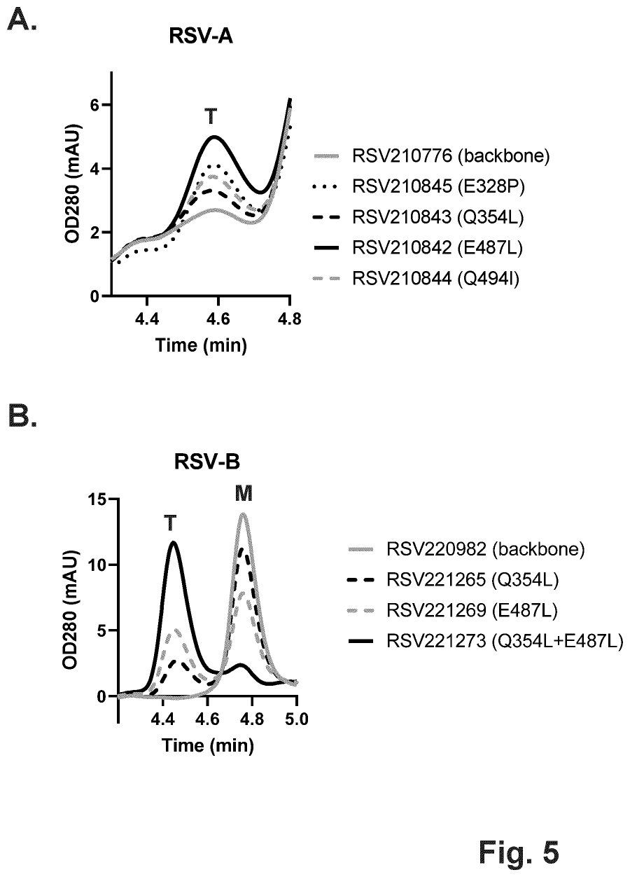

RSV-A F trimer expression was increased by the introduction of E328P (RSV210845), Q354L (RSV210843), Q494I (RSV210844), and in particular by E487L (RSV210842) (FIG. 5A). Introduction of Q354L (RSV221265) and E487L (RSV221269) in RSV-B F both resulted in detectable trimer expression (FIG. 5B). The combinatorial substitution of Q354L+E487L (RSV221273) significantly enhanced RSV-B F trimer yield and reduced monomer expression (FIG. 5B), demonstrating that head domain stabilization improved RSV F ectodomain trimer expression.

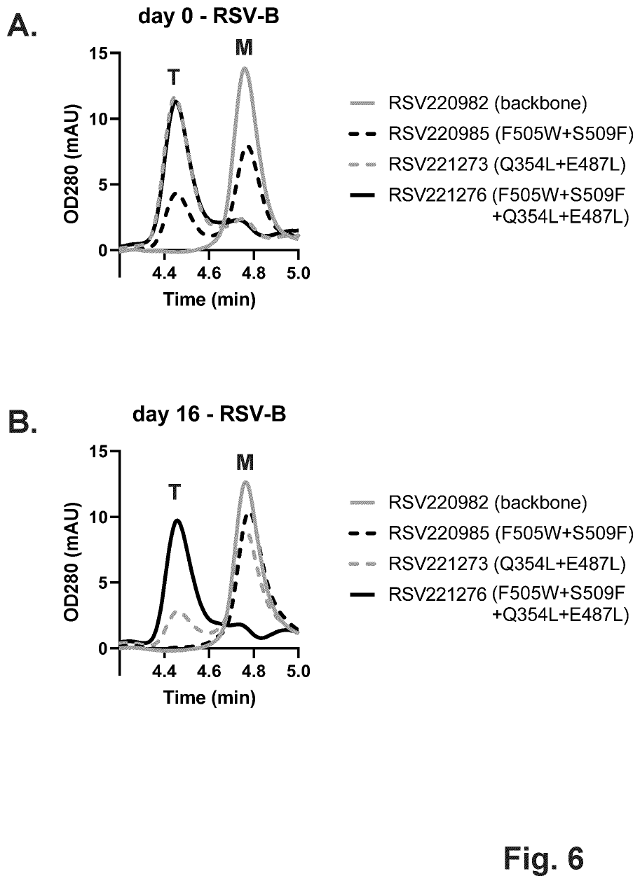

The stability of RSV-B F ectodomain trimers was assessed by analytical SEC of crude cell-culture supernatant after 16 days storage at 4° C. These data demonstrated that although initially significant trimer expression was detected for RSV221273 carrying head domain substitutions Q354L+E487L (FIG. 5B, FIG. 6A), these trimers were strongly reduced after 16 days of storage at 4° C. (FIG. 6B). Likewise, HR2 stabilization F505W+S509F in RSV220985 was sufficient to detect RSV-B F trimer expression at day of harvest (FIG. 3B, FIG. 6A), but did not lead to stable trimer expression after storage at 4 degrees for 16 days (FIG. 6B). The combinatorial stabilization of the RSV-B F head domain and HR2 region, however, did yield stable trimer expression (FIG. 6B, RSV221276), indicating that stabilization of both regions contributes to the overall trimer ectodomain stability in the absence of a heterologous trimerization domain. These results are in line with improved heat stability that was observed for RSV-A F with stabilization of both the head domain and the HR2 region (RSV210789, FIG. 4).

### Example 4. Effect of HR2 Length and Stabilization on Trimerization of RSV-A and RSV-B F in the Absence of a Heterologous Trimerization Domain

The impact of the position of the truncation (i.e. length of the HR2) on RSV-A and RSV-B F trimer ectodomain expression was assessed in various backbones. Elongation of HR2 involved the addition of 11 amino acids (HNVNAGKSTTN) from full-length RSV-A and RSV-B F sequence to the frequently used variants that were truncated at position 513. Variants with ‘short’ HR2 (truncated at position 513) were compared with ‘long’ HR2 variants (truncated at position 524) by assessing trimer expression in cell culture supernatant as described in example 1.

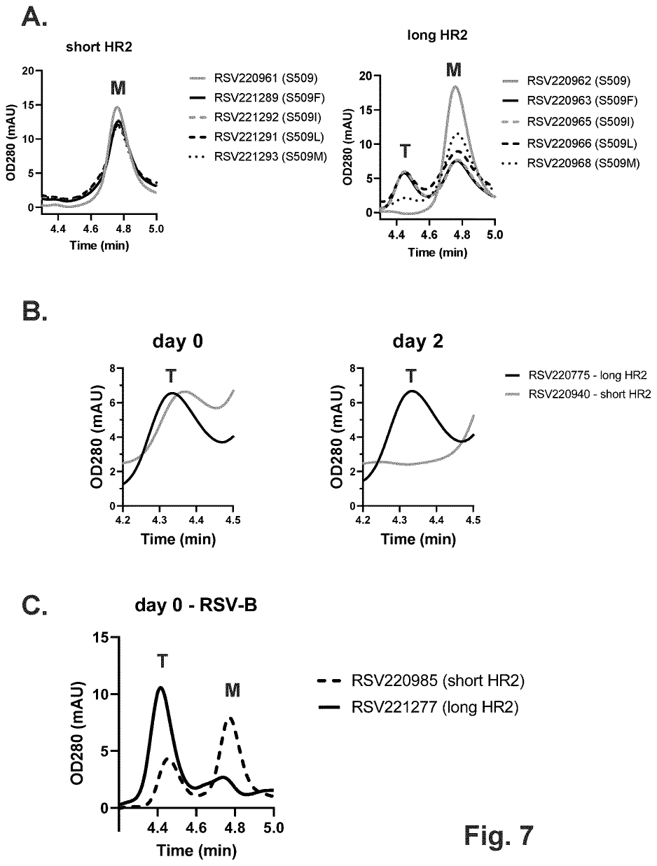

Improved trimer expression by S509 substitutions in the RSV-A F ectodomain was measured in long HR2 variants, carrying substitutions N67I, S215P and D486N (RSV220963, RSV220965, RSV220966, RSV220968), but could not be detected in comparable variants expression a shorter HR2 region (RSV221289, RSV221292, RSV221291, RSV221293) (FIG. 7A). Moreover, stability of RSV-A F trimer expression was enhanced in a variant with long HR2, carrying P101Q, S215P, D486N, D489Y, F505W, and S509F (RSV220775), compared to equivalent RSV220940 with short HR2, wherein RSV-A F trimer could not be detected after two days storage at 4° C. (FIG. 7B). HR2 elongation likewise had a positive impact on RSV-B F trimer ectodomain expression in a variant carrying P101Q, 1152M, L203I, S215P, D486N, D489Y, F505W, and S509F (RSV221277). Trimer expression was significantly enhanced, and monomer expression was reduced compared to the same variant with short HR2 (RSV220985) (FIG. 7C). As expected, due to the longer HR2 (i.e. addition of 11 amino acids), the trimer of RSV221277 eluted slightly earlier than the short HR2 variant (FIG. 7C).

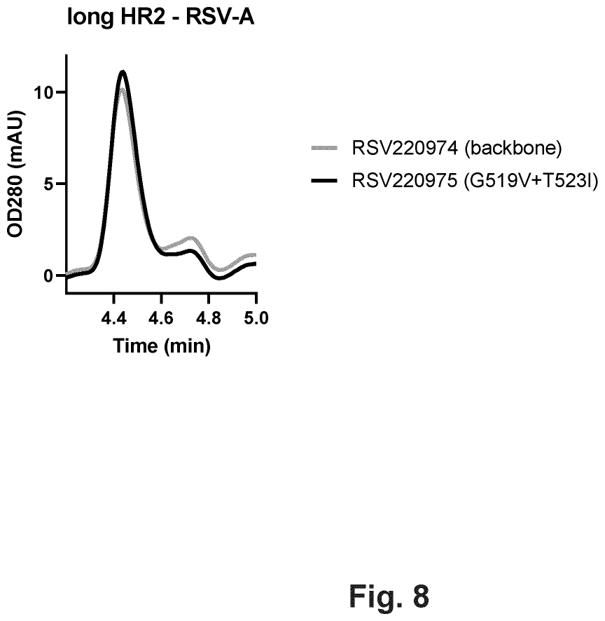

The elongated HR2 region was further stabilized by introduction of G519V and T523I substitutions. In a stabilized long HR2 RSV-A F ectodomain variant without a heterologous trimerization domain, and with P101Q, S215P, Q354L, D486N, E487L, D489Y, F505W, and S509F (RSV220974), introduction of G519V+T523I increased trimer expression (RSV220975) (FIG. 8A).

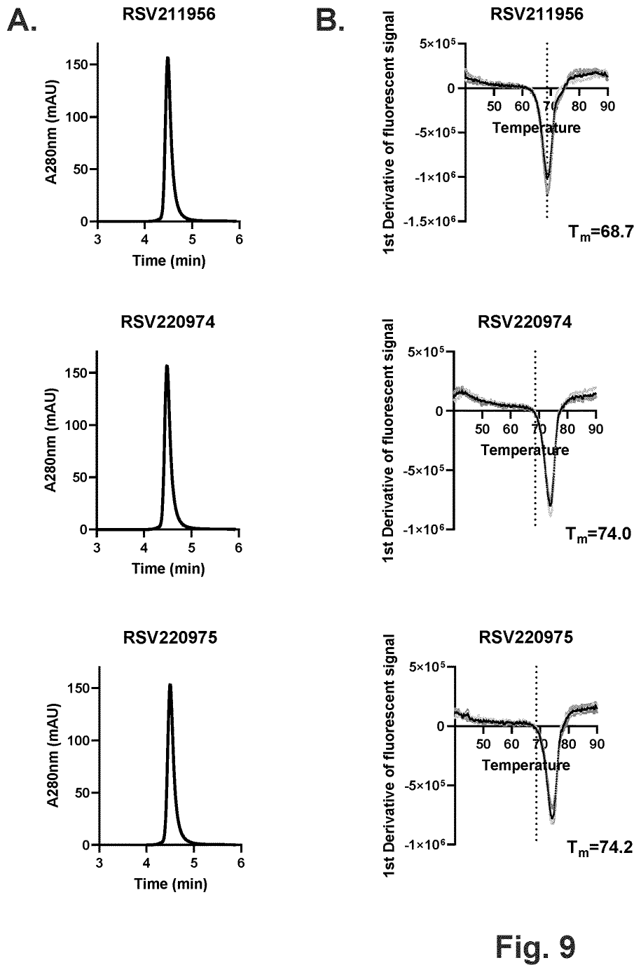

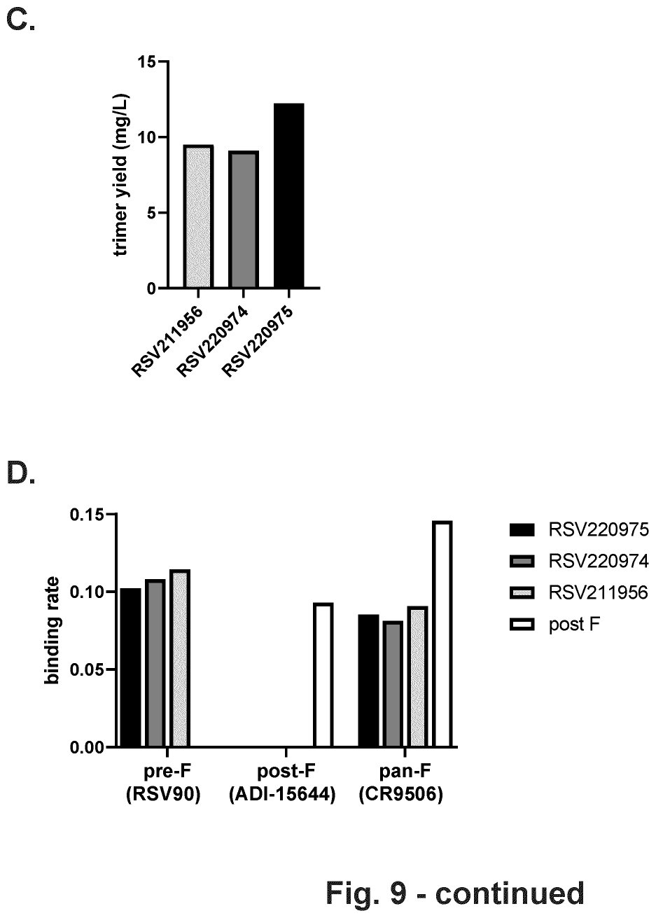

Stabilized RSV-A F variants with either a short HR2 (RSV211956), long HR2 (RSV220974), or long stabilized HR2 domain (RSV220975) were purified. None of the variants had a foldon trimerization domain or a C-tag purification tag, and all carried stabilizing mutations P101Q, S215P, Q354L, D486N, E487L, D489Y, F505W, and S509F. Plasmids were transiently transfected in Expi293F cells using ExpiFectamine (Life Technologies) according to the manufacturer's instructions and cultured for 5 days at 37° C. and 10% CO₂. The culture supernatant was harvested and spun for 10 minutes at 600 g to remove cells and cellular debris, then sterile-filtered using a 0.22 μm vacuum filter. RSV-A F proteins were purified using a two-step purification protocol including ion exchange (Cation) purification at pH 5.0 and polishing via size exclusion chromatography using a Superdex 200 16/600 pg column. The trimeric fraction was pooled and further characterized by analytical SEC-MALS using an ultra-high-performance liquid chromatography system (Vanquish, Thermo Scientific) and μDAWN TREOS instrument (Wyatt) coupled to an Optilab μT-rEX Refractive Index Detector (Wyatt), in combination with an in-line Nanostar DLS reader (Wyatt). Protein was loaded onto a Unix-C SEC-300 15 cm column (Sepax Technologies) with the corresponding guard column (Sepax Technologies) equilibrated in running buffer (150 mM sodium phosphate, 50 mM NaCl, pH 7.0) at 0.35 mL/min. Analytical SEC data was analyzed using Chromeleon 7.2.8.0 software package, and conformation, hydrodynamic radius and molecular weight of F trimers was calculated by Astra software and compared to the calculated weight, confirming a trimeric conformation (FIG. 9A)

Melting temperature (Tm₅₀) of purified RSV-A F trimers was determined by differential scanning fluorimetry (DSF). To this end, the fluorescent emission of Sypro Orange Dye (ThermoFisher Scientific) added to RSV-A F protein in solution was monitored. The measurement was performed with a starting temperature of 25° C. and a final temperature of 95° C. (54° C. increase per hour). Melting curves were measured using a ViiA7 real-time PCR machine (Applied Biosystems), and Tm50 values were derived from the negative first derivative as described previously (Rutten et al. (2020) Cell Rep 30:4540-4550). RSV220974 with a long HR2 had a higher melting temperature of 74.0° C. compared with a melting temperature of 68.7° C. for RSV211956 with a short HR2, confirming improved stability by elongating the HR2 region in RSV-A F ectodomains (FIG. 9B). Substitutions G519V+T523I in RSV220975 did not impact the melting temperature compared to RSV220975 (74.2° C. and 74.0° C., respectively), but did increase trimer yield (FIG. 9C).

The prefusion conformation of purified RSV-A F proteins was confirmed in biolayer interferometry (BLI) measurements using quantitative Octet with RSV-F A prefusion-, postfusion-, and pan-specific monoclonal antibodies RSV90 (Mousa et al. (2017) Nat Microbiol, 2:16271), ADI-15644 (Gilman et al. (2016) Sci Immunol, 1: eaaj1879), and CR9506 (comprising the heavy and light chain variable region as disclosed in WO20/099383), respectively. Antibodies were immobilized to anti-human IgG sensors at a concentration of 5 μg/ml, and initial binding rate of RSV-F A at 20 μg/ml during 300 seconds association was plotted (FIG. 9D). Postfusion RSV-A F protein (RSV150043) was included as an assay control. Binding to pan-specific CR9506 was confirmed for all four RSV-A F proteins, while postfusion F-specific binding was only detected for postfusion F protein. Conversely, prefusion F-specific binding was only detected for the stabilized RSV-A F variants (FIG. 9D).

### Example 5. Purification, Characterization, and Immunogenicity of Stabilized RSV-A F Variants with and without a Heterologous Trimerization Domain

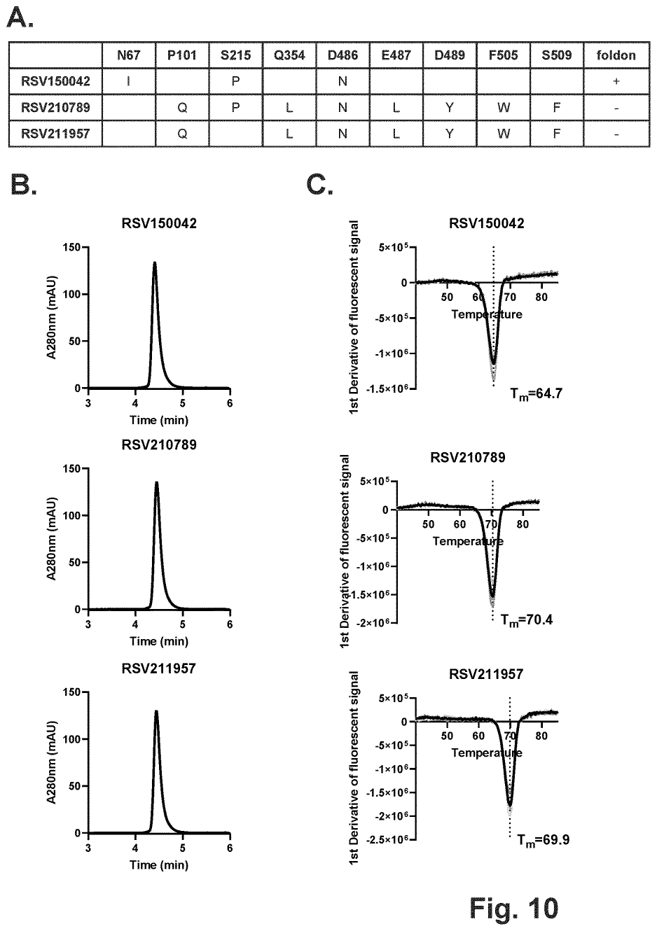

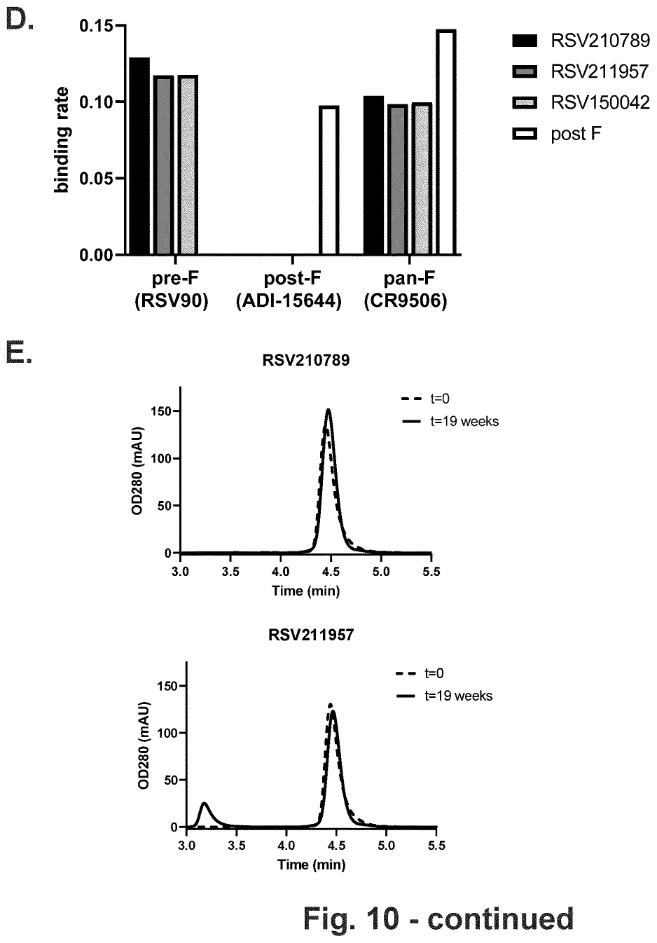

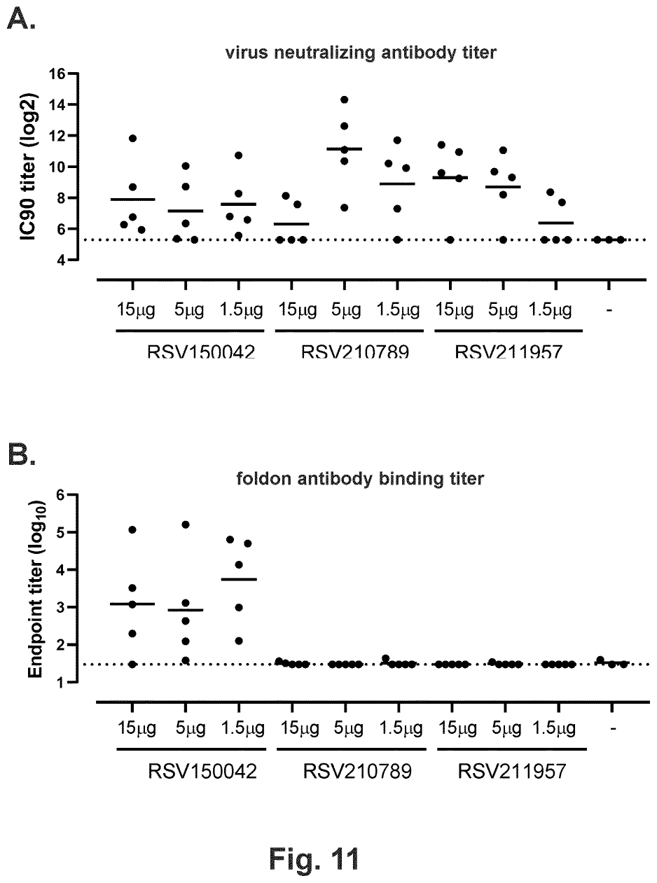

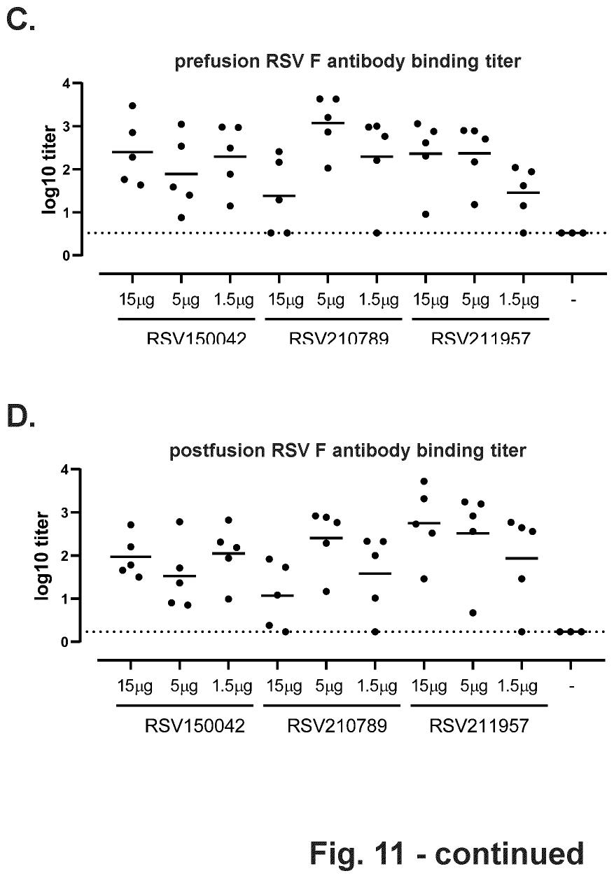

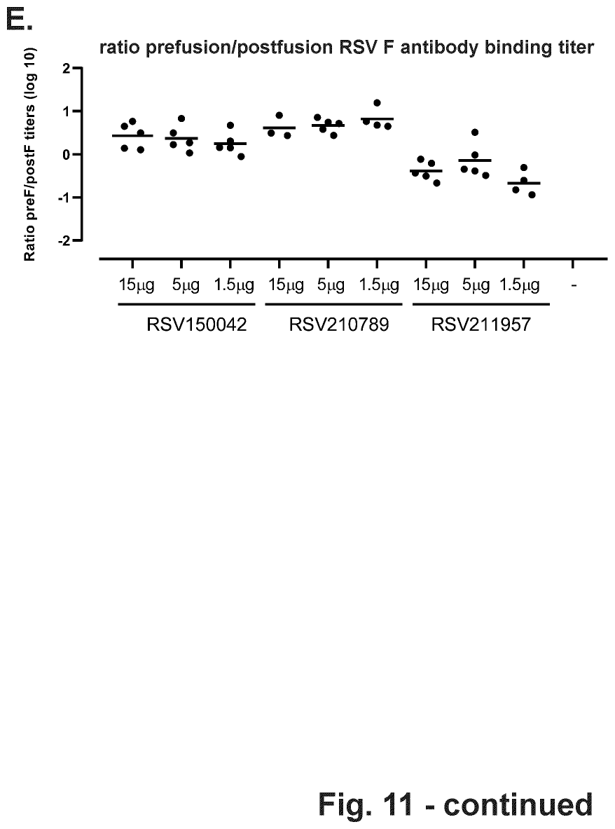

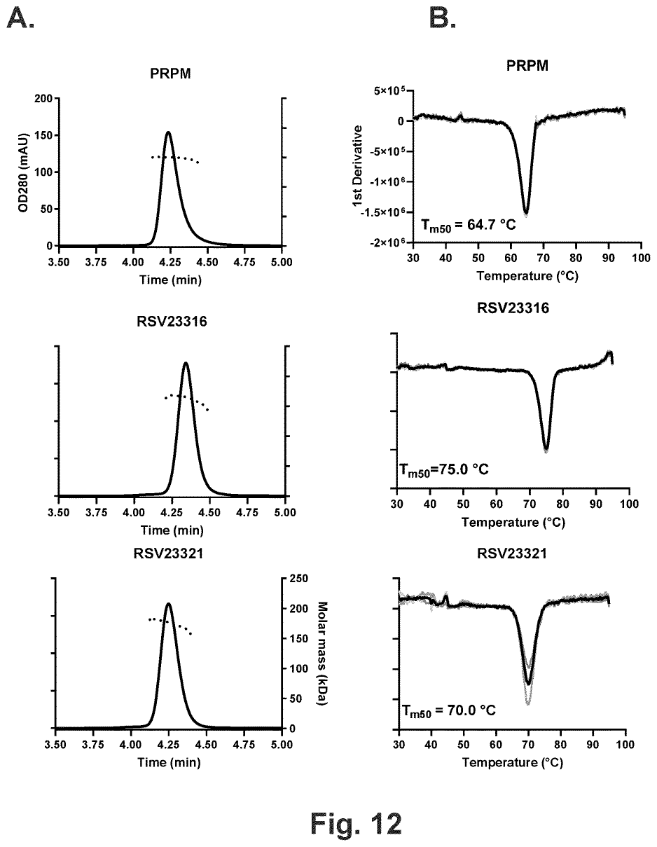

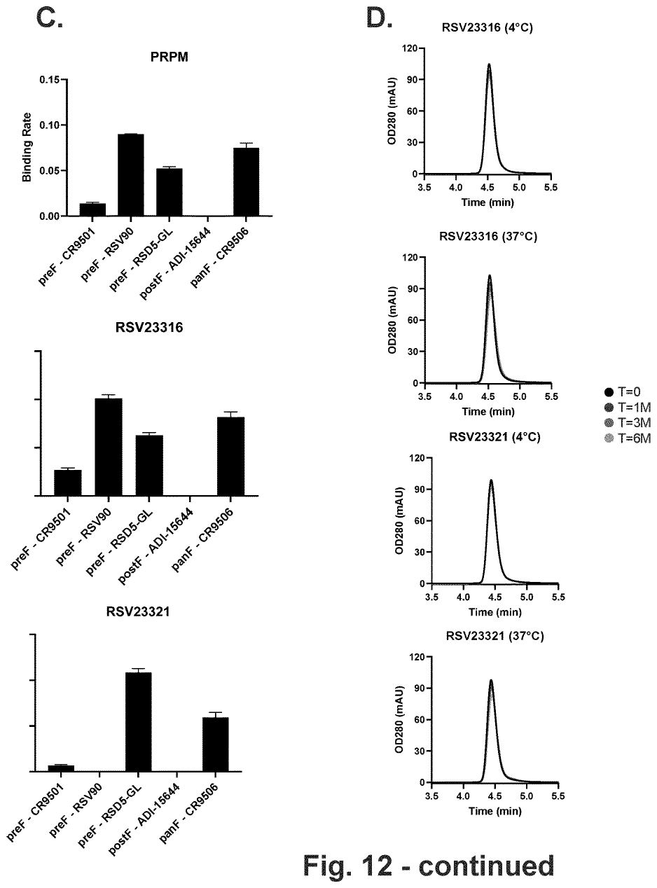

RSV-A F variants with (RSV150042) and without (RSV210789 and RSV211957) a foldon trimerization domain and with stabilizing mutations as listed in FIG. 10A were transiently transfected in Expi293F cells using ExpiFectamine (Life Technologies) according to the manufacturer's instructions and cultured for 5 days at 37° C. and 10% CO₂. The culture supernatant was harvested and spun for 10 minutes at 600 g to remove cells and cellular debris, then sterile-filtered using a 0.22 μm vacuum filter. RSV-A F protein with a foldon trimerization domain (RSV150042) was purified using a two-step purification protocol including ion exchange (Cation) purification at pH 5.0 and polishing via size exclusion chromatography using a Superdex 200 16/600 pg column. RSV-A F proteins without a foldon trimerization domain and with a C-tag purification tag (RSV210789 and RSV211957) were purified using a two-step purification protocol including CaptureSelect™ C-tag affinity column and polishing via size exclusion chromatography using a Superdex 200 16/600 pg column. The trimeric fraction was pooled and further characterized by analytical SEC-MALS using an ultra-high-performance liquid chromatography system (Vanquish, Thermo Scientific) and μDAWN TREOS instrument (Wyatt) coupled to an Optilab μT-rEX Refractive Index Detector (Wyatt), in combination with an in-line Nanostar DLS reader (Wyatt). Protein was loaded onto a Unix-C SEC-300 15 cm column (Sepax Technologies) with the corresponding guard column (Sepax Technologies) equilibrated in running buffer (150 mM sodium phosphate, 50 mM NaCl, pH 7.0) at 0.35 mL/min. Analytical SEC data was analyzed using Chromeleon 7.2.8.0 software package, and conformation, hydrodynamic radius and molecular weight of F trimers was calculated by Astra software and compared to the calculated weight, confirming a trimeric conformation (FIG. 10B).

Melting temperature (Tm50) of purified RSV-A F trimers was determined by differential scanning fluorimetry (DSF). To this end, the fluorescent emission of Sypro Orange Dye (ThermoFisher Scientific) added to RSV-A F protein in solution was monitored. The measurement was performed with a starting temperature of 25° C. and a final temperature of 95° C. (54° C. increase per hour). Melting curves were measured using a ViiA7 real-time PCR machine (Applied Biosystems), and Tm50 values were derived from the negative first derivative as described previously (Rutten et al. (2020) Cell Rep 30:4540-4550). RSV150042 with a foldon trimerization domain had a lower melting temperature of 64.7° C. compared with RSV210789 and RSV211957 (70.4° C. and 69.9° C., respectively) without a trimerization domain and with stabilizing amino acid substitutions according to the invention (FIG. 10C).

The prefusion conformation of purified RSV-A F proteins was confirmed in biolayer interferometry (BLI) measurements using quantitative Octet with RSV-F A prefusion-, postfusion-, and pan-specific monoclonal antibodies RSV90 (supra), ADI-15644 (supra) and CR9506 (supra), respectively. Antibodies were immobilized to anti-human IgG sensors at a concentration of 5 μg/ml, and initial binding rate of RSV-F A at 20 μg/ml during 300 seconds association was plotted (FIG. 10D). Postfusion RSV-A F protein (RSV150043) was included as an assay control. Binding to pan-specific CR9506 was confirmed for all four RSV-A F proteins, while postfusion F-specific binding was only detected for postfusion F protein. Conversely, prefusion F-specific binding was only detected for the stabilized RSV-A F variants, either with or without a foldon trimerization domain (FIG. 10D).

The storage stability at 4° C. was assessed for purified RSV-A F variants without a heterologous trimerization domain. RSV210789 remained stable for 19 weeks storage at 4° C., while some aggregates were detected for RSV211957, which eluted between 3-3.5 minutes retention time (FIG. 10E). The presence of aggregates in RSV211957 indicates that S215P removal is suboptimal for RSV-A F protein stability. It was thus shown that without the S215P mutation the protein can still transition towards a post-fusion conformation.

The immunogenicity of purified RSV-A F proteins RSV150042, RSV210789 and RSV211957 was assessed by immunizing naïve mice (female Balb/c) intramuscularly with either 15, 5 or 1.5 μg RSV-A F protein variants (n=5 per dose). As controls, mice were intramuscularly injected with formulation buffer (n=3).

Serum samples were isolated two weeks after the second immunization (week 6) and used to measure virus neutralizing antibody titers (VNT) by a firefly luciferase (FFL) reporter-based assay for RSV A CL57, RSV-A prefusion F and postfusion F binding antibodies titers and foldon binding antibody titers by ELISA.

RSV-A prefusion F and postfusion F binding antibody titers (FIG. 11C, D) as well as RSV CL57 VNT (FIG. 11A) were induced by RSV150042 and by RSV210789 and RSV211957 (the two stabilized, trimeric prefusion RSV-A F proteins according to the present invention without a heterologous trimerization domain) without any obvious different levels across doses induced by the preF protein variants. The preF/postF binding antibody titer ratio was calculated for samples with at least one titer above LLOD (FIG. 11E). RSV150042 and RSV210789 showed a similar preF/postF binding antibody ratio, while RSV211957 showed a more postF-skewed preF/postF binding antibody ratio. It was confirmed that the two stable, trimeric prefusion RSV-A F protein without a foldon trimerization domain did not induce foldon binding antibody titers while RSV150042 did induce foldon binding antibody titers (FIG. 11B).

In summary, these data demonstrate that stable, trimeric prefusion RSV-A F protein without a heterologous trimerization domain can be produced, and that this protein is immunogenic in mice.

EXAMPLE 6: Purification, characterization, and immunogenicity of stabilized foldon-free and tagless RSV-A and RSV-B preF proteinsNext, we generated stabilized, foldon-free and tagless RSV-A and RSV-B preF proteins based on the sequence of recently circulating strains. A 524 amino acid-long full ectodomain sequence was used, containing the head substitutions P101S, 1152M, S215P, Q354L, D486N, E487L and D489Y, as well as the HR2-stem substitutions F505W, S509F, G519V and T523I. Stabilized RSV-A (RSV23316) and RSV-B (RSV23321) F trimers were expressed in Expi293F cells, purified (IEX followed by SEC polishing) and characterized, and were compared to a benchmark RSV-A preF protein (‘PRPM’) with a foldon trimerization domain (Krarup et al. 2015). On analytical SEC the proteins eluted at the expected retention time (Rt) with RSV23316 and RSV23321 eluting at a slightly longer Rt than PRPM due to the absence of foldon (FIG. 12A). Foldon-free RSV-A F protein RSV23316 had a melting temperature of 75° C.; an increase of >10° C. compared to the benchmark foldon-containing RSV-A F variant PRPM (FIG. 1B). Foldon-free RSV-B protein, RSV23321, had an intermediate melting point of 70° C. (FIG. 12B). The antigenicity profile of all three proteins was representative of a prefusion RSV F conformation, showing binding of prefusion-specific CR9501, RSV90 and RSD5-GL antibodies in the absence of binding of post-fusion specific ADI-15644 antibody (FIG. 12C). As expected, RSV90 does not bind the F protein of RSV-B due to mutations in the RSV90 epitope (Mousa et al. 2017). Both foldon-free preF proteins demonstrated an excellent stability profile, retaining a trimeric conformation during long-term storage up to six months at 4° C. and 37° C. (FIG. 12D).

TABLE 1
Standard amino acids, abbreviations and properties

| | 3- | 1- | Side chain | |
|---|---|---|---|---|
| Amino Acid | Letter | Letter | polarity | Side chain charge (pH 7.4) |
| alanine | Ala | A | non-polar | Neutral |
| arginine | Arg | R | polar | Positive |
| asparagine | Asn | N | polar | Neutral |
| aspartic acid | Asp | D | polar | Negative |
| cysteine | Cys | C | non-polar | Neutral |
| glutamic acid | Glu | E | polar | Negative |
| glutamine | Gln | Q | polar | Neutral |
| glycine | Gly | G | non-polar | Neutral |
| histidine | His | H | polar | Positive (10%) neutral(90%) |
| isoleucine | Ile | I | non-polar | Neutral |
| leucine | Leu | L | non-polar | Neutral |
| lysine | Lys | K | polar | Positive |
| methionine | Met | M | non-polar | Neutral |
| phenylalanine | Phe | F | non-polar | Neutral |
| proline | Pro | P | non-polar | Neutral |
| serine | Ser | S | polar | Neutral |
| threonine | Thr | T | polar | Neutral |
| tryptophan | Trp | W | non-polar | Neutral |
| tyrosine | Tyr | Y | polar | Neutral |
| valine | Val | V | non-polar | Neutral |

1. Respiratory syncytial virus (RSV) F protein, comprising a head region and a stem region, wherein the protein comprises at least one amino acid mutation in the stem region relative to the amino acid sequence of a wild-type RSV F protein, wherein the at least one mutation in the stem region comprises a mutation of the amino acid residue at position 509 into F, I or L, and wherein amino acid positions are numbered according to the numbering of the amino acid residues in SEQ ID NO:1.

2. Protein according to claim 1, wherein the protein further comprises at least one amino acid mutation in the head region relative to the amino acid sequence of a wild-type RSV F protein and at least one amino acid mutation in the stem region, wherein the at least one mutation in the stem region comprises a mutation of the amino acid residue at position 509 into F, I or L.

3. Protein according to claim 2, wherein the at least one amino acid mutations in the head region is a mutation of the amino at residue 215 into P or a mutation of the amino acid residue at position 486 into N.

4. Protein according to claim 2, wherein the protein comprises at least one additional mutation in the head region.

5. Protein according to claim 4, wherein the protein comprises at least two amino acid mutations in the head region relative to the amino acid sequence of a wild-type RSV F protein and at least one amino acid mutation in the stem region, wherein the at least one mutation in the stem region comprises a mutation of the amino acid residue at position 509 into F, I or L.

6. Protein according to claim 4, comprising at least three amino acid mutations in the head region relative to the amino acid sequence of a wild-type RSV F protein and at least one amino acid mutation in the stem region, wherein the at least one mutation in the stem region comprises a mutation of the amino acid residue at position 509 into F, I or L.

7. Protein according to claim 1, wherein the RSV F protein is an F protein of an RSV A subtype.

8. Protein according to claim 1, wherein the RSV F protein is an F protein of an RSV B subtype.

9. Protein according to claim 4, wherein the amino acid at position 101 is not P.

10. Protein according to claim 1, wherein the at least one additional mutation in the head region is selected from the group consisting of a mutation of the amino acid residue at position 101 into Q, S, T, A, or G, a mutation of the amino acid residue at position 328 into P, a mutation of the amino acid residue at position 354 into L, a mutation of the amino acid at position 487 into L, a mutation of the amino acid residue at position 489 into Y, a mutation of the amino acid residue at position 494 into I, a mutation of the amino acid residue at position 519 into V and a mutation of the amino acid residue 523 into I.

11. Protein according to claim 1, wherein the at least one additional mutation in the head region is selected from the group consisting of a mutation of the amino acid residue at position 101 into Q, S, T, A, or G, a mutation of the amino acid residue at position 152 into M, a mutation of the amino acid residue at position 203 into I, a mutation of the amino acid residue at position 354 into L, a mutation of the amino acid at position 487 into L, a mutation of the amino acid residue at position 489 into Y.

12. Protein according to claim 1, wherein the at least one mutation of the amino acid residue in the stem region at position 509 is a mutation into F.

13. Protein according to claim 1, further comprising a second mutation in the stem region, wherein said second mutation is a mutation of the amino acid residue at position 505 into W.

14. Protein according to claim 5, wherein the protein has been truncated after the amino acid residue 524.

15. Protein according to claim 6, wherein the protein has been truncated at the C-terminus after amino acid residue 513.

16. Protein according to claim 14, wherein the protein does not comprise a heterologous trimerization domain.

17. Protein according to claim 1, wherein the protein is trimeric.

18. Protein according to claim 1, comprising an amino acid sequence selected from the group consisting of SEQ ID NO: 9, 11, 13, 17-29, 34, and 38-39, or an amino acid sequence with at least 90% amino acid sequence identity, or a fragment thereof.

19. Fragment of a protein according to claim 1.

20. Nucleic acid encoding a protein according to claim 1.

21. Nucleic acid according to claim 20, wherein the nucleic acid is DNA or RNA.

22. Nucleic acid according to claim 21, wherein the RNA is mRNA, modified mRNA, self-replicating RNA, or circular mRNA.

23. Nucleic acid according to claim 20, encoding a protein comprising an amino acid sequence selected from the group consisting of SEQ ID NO: 9, 11, 13, 17-29, 34, and 38-39, or an amino acid sequence with at least 90% amino acid sequence identity, or a fragment thereof.

24. Vector comprising a nucleic acid according to claim 20.

25. Composition comprising a protein according to claim 20.

26. A method for vaccinating a subject against RSV, the method comprising administering to the subject a composition according to claim 25.

27. A method for preventing infection and/or replication of RSV in a subject, comprising administering to the subject a composition according to claim 25.

28. An isolated host cell comprising a nucleic acid according to claim 20.


SEQUENCE LISTING (44 sequences)

SEQ ID NO: 1 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 2 - protein, 544 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLSAIGGYIPEAPRDGQAYVRKDGEWVLLSTFL

SEQ ID NO: 3 - protein, 513 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELL

SEQ ID NO: 4 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLHNVNAGKSTTN

SEQ ID NO: 5 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKYDELLHNVNAGKSTTN

SEQ ID NO: 6 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKVDELLHNVNAGKSTTN

SEQ ID NO: 7 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKMDELLHNVNAGKSTTN

SEQ ID NO: 8 - protein, 513 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKMDELL

SEQ ID NO: 9 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKFDELLHNVNAGKSTTN

SEQ ID NO: 10 - protein, 513 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKFDELL

SEQ ID NO: 11 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKIDELLHNVNAGKSTTN

SEQ ID NO: 12 - protein, 513 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKIDELL

SEQ ID NO: 13 - protein, 524 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKLDELLHNVNAGKSTTN

SEQ ID NO: 14 - protein, 513 aa, synthetic construct
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKLDELL

SEQ ID NO: 15 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAFIRKSDELLGGSEPEA

SEQ ID NO: 16 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAWIRKSDELLGGSEPEA

SEQ ID NO: 17 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAFIRKFDELLGGSEPEA

SEQ ID NO: 18 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAWIRKFDELLGGSEPEA

SEQ ID NO: 19 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAWIRKFDELLGGSEPEA

SEQ ID NO: 20 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKPGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAWIRKFDELLGGSEPEA

SEQ ID NO: 21 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAWIRKFDELLGGSEPEA

SEQ ID NO: 22 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISIVNEKINQSLAWIRKFDELLGGSEPEA

SEQ ID NO: 23 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFDASISQVNEKINQSLAWIRKFDELLGGSEPEA

SEQ ID NO: 24 - protein, 524 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFYASISQVNEKINQSLAWIRKFDELLHNVNAGKSTTN

SEQ ID NO: 25 - protein, 513 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFYASISQVNEKINQSLAWIRKFDELL

SEQ ID NO: 26 - protein, 513 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAWIRKFDELL

SEQ ID NO: 27 - protein, 524 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAWIRKFDELLHNVNAGKSTTN

SEQ ID NO: 28 - protein, 524 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAWIRKFDELLHNVNAVKSTIN

SEQ ID NO: 29 - protein, 520 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNLFYASISQVNEKINQSLAWIRKFDELLGGSEPEA

SEQ ID NO: 30 - protein, 544 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFL

SEQ ID NO: 31 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELL

SEQ ID NO: 32 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAWIRRSDELL

SEQ ID NO: 33 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRFDELL

SEQ ID NO: 34 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAWIRRFDELL

SEQ ID NO: 35 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPLADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELL

SEQ ID NO: 36 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNLFYASISQVNEKINQSLAFIRRSDELL

SEQ ID NO: 37 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPLADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNLFYASISQVNEKINQSLAFIRRSDELL

SEQ ID NO: 38 - protein, 513 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPLADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNLFYASISQVNEKINQSLAWIRRFDELL

SEQ ID NO: 39 - protein, 524 aa, synthetic construct
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAWIRRFDELLHNVNTGKSTTN

SEQ ID NO: 40 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALQLTNKAVVSLSNGVSVLTSRVLDLKNYINNQLLPMVNRQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 41 - protein, 8 aa, synthetic construct
WSHPQFEK

SEQ ID NO: 42 - protein, 4 aa, synthetic construct
EPEA

SEQ ID NO: 43 - skipped

SEQ ID NO: 44 - protein, 524 aa, synthetic construct
MELLILKANAITTILTAVTFCFASGQNITEEFYQSTCSAVSKGYLSALRTGWYTSVITIELSNIKETKCNGTDTKVKLIKQELDKYKNAVTELQLLMQNTSAVNNRARREAPQYMNYTINTTKNLNVSISKKRKRRFLGFLLGVGSAIASGMAVSKVLHLEGEVNKIKNALQLTNKAVVSLSNGVSVLTNRVLDLKNYINNQLLPMVNRQNCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPLADTCKVQSNRVFCDTMNSLTLPSEVSLCNTDIFNPKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNLFYASISQVNEKINQSLAWIRRFDELLHNVNTVKSTIN